(19) United States
(12) Patent Application Publication
(10) Pub. No.: US 2017/0296591 A1
(43) Pub. Date: Oct. 19, 2017

(54) CARDIOSPHERE-DERIVED CELLS (CDCS) AS THERAPEUTIC AGENTS FOR PULMONARY HYPERTENSION

(71) Applicant: Cedars-Sinai Medical Center, Los Angeles, CA (US)

(72) Inventors: Eduardo Marban, Santa Monica, CA (US); Michael I. Lewis, Covina, CA (US)

(73) Assignee: Cedars-Sinai Medical Center, Los Angeles, CA (US)

(21) Appl. No.: 15/515,979

(22) PCT Filed: Oct. 2, 2015
(86) PCT No.: PCT/US15/53822

§ 371 Date: Mar. 30, 2017

Related U.S. Application Data
(60) Provisional application No. 62/059,485, filed on Oct. 3, 2014.

(51) Int. Cl. A61K 35/34; A61K 9/00

(52) U.S. Cl. CPC A61K 35/34; A61K 9/0019

(57) ABSTRACT

Described herein are compositions and techniques for treatment of disease and conditions such as pulmonary arterial hypertension (PAH). Unlike palliative or preventive measures that do not address the abnormal vasculature causing onset of right ventricular compensation the use of stem cell-based therapy can directly impact the microvascular pathology in PAH, thereby reversing the course of the disease.

### FIELD OF THE INVENTION

Described herein are compositions and techniques related to use of cardiosphere-derived cells in pulmonary arterial hypertension (PAH).

### BACKGROUND

Pulmonary arterial hypertension (PAH) is a progressive and fatal condition characterized by marked narrowing or obstruction of small pulmonary arterioles throughout the lungs. These occlusions in the vasculature of the lungs, leads to increased pulmonary vascular resistance facing the right ventricle as it attempts to pump blood through the lungs. Studies have shown PAH is connected to decreased prostacyclin and nitric oxide in the lining of the small lung vessels. These factors normally dilate the vessel and prevent cellular proliferation in the vessel wall. In addition, PAH is connected to increased levels of endothelin-1, a substance which has the opposite effects. As a result, therapeutic approaches have relied on drugs to replace the “good factors” or block the “bad factor”, many which have been developed and used since 1995. Several delivery forms of prostacyclin analogues, agents that augment the downstream effects of nitric oxide and endothelin receptor antagonists, have been used to treat patients with PAH. These treatment approaches have delivered improved survival effort tolerance, and quality of life for those afflicted with PAH. However, they are not at all curative and PAH progresses unimpeded despite the aforementioned treatment, and patients may succumb to right heart failure as a result. In fact, progressive right heart decompensation ensues, despite apparent clinical progress.

A chief limitation of existing therapies is that they fail to reverse the underlying pathological changes in the small pulmonary arterioles that are the root of the disease. To date there are no specific treatments that address the many pathobiologic mechanisms underlying right ventricle dysfunction in patients with PAH. A highly promising avenue has been use of stem cells introduced directly into the site of injury, wherein such stem cells can be introduced directly into the pulmonary vasculature or intravenously via infusion. Importantly, various studies observe that following stem cell administration, the right heart ventricle experience lowered resistance during blood ejection. As the work of the right heart is reduced, lower pulmonary pressures and reduced right ventricular wall thickness suggest better cardiac performance. Additional stem cell related PAH studies have shown that the infusion of cardiac stem cells, such as cardiosphere-derived cells (CDCs) are also capable of significantly reduced pulmonary pressures and right ventricular wall thickness. At least one stem cell based therapy for PAH has reached the clinic.

Described herein are compositions and techniques related to generation and therapeutic application of CDCs in PAH, wherein such stem cells are capable in not only preventing or ameliorating disease and/or conditions such as PAH, but actually capable of treating PAH itself via regeneration and repair of damaged microvasculature.

### SUMMARY OF THE INVENTION

Described herein is a method of treatment, including selecting a subject in need of treatment for a heart related disease and/or condition, and administering a composition including cardiosphere-derived cells (CDCs), wherein the administration of the composition treats the subject. In other embodiments, the heart related disease and/or condition includes pulmonary hypertension (PH) or pulmonary arterial hypertension (PAH). In other embodiments, the PAH is idiopathic. In other embodiments, the PAH is associated. In other embodiments, the subject has a chronic disease and/or condition. In other embodiments, the single dose is administered multiple times to the subject. In other embodiments, administering a composition includes intra-arterial infusion. In other embodiments, administering a composition includes intravenous infusion. In other embodiments, administering a composition includes injection. In other embodiments, injection includes percutaneous injection. In other embodiments, injection includes injection into heart muscle. In other embodiments, treatment results in reduced ventricular wall thickness. In other embodiments, treatment results in reduced pulmonary pressure or systolic pressure.

Further described herein is a method of treatment, including selecting a subject in need of treatment for a heart related disease and/or condition, and administering a composition including stem cells, progenitors and/or precursors to the subject, wherein the administration of the composition treats the subject. In other embodiments, the heart related disease and/or condition includes pulmonary hypertension (PH) or pulmonary arterial hypertension (PAH).
In other embodiments, the PAH is idiopathic. In other embodiments, the PAH is associated.
In other embodiments, the stem cells, progenitors and/or precursors comprise cardiosphere-derived cells (CDCs). In other embodiments, the subject has a chronic disease and/or condition.
In other embodiments, the single dose is administered multiple times to the subject. In other embodiments, administering a composition includes intra-arterial infusion. In other embodiments, administering a composition includes intravenous infusion. In other embodiments, administering a composition includes injection. In other embodiments, injection includes percutaneous injection. In other embodiments, injection includes injection into heart muscle. In other embodiments, treatment results in reduced ventricular wall thickness. In other embodiments, treatment results in reduced pulmonary pressure or systolic pressure.

### BRIEF DESCRIPTION OF FIGURES

FIG. 1. Therapeutic Effects of Cardiosphere-Derived Cells in PAH Animal Model. Animals were divided into control, monocrotaline sham infusion (i.e., no cells) and monocrotaline with cardiosphere-derived cells (+CDCs). Echo studies and final hemodynamic studies indicated that a single dose of 2 million CDCs, infused intravenously, markedly attenuate the progression of PAH in the monocrotaline rat model, as shown by the decrease systolic pressure in the right ventricle when CDCs are administered. Right ventricular systolic pressure (RVSP, upper panel) and Fulton Index (right ventricle/[left ventricle+septum], RV/(LV+S); lower panel) in healthy control and animals with PAH 35 days following monocrotaline subcutaneous injection (60 mg/kg) and intravenous infusion of phophase buffered saline (PBS; sham surgery) or 2 million CDCs in PBS on day 14. Values are means±SEM. * significantly different from control (CTL) # significantly different from PBS.

FIG. 2. Differential Expression of microRNAs in CDC Exosomes. (A) MicroRNA analysis of CDC-derived exosomes demonstrate the differential cargo contents of exosomes based on parental cellular origin. Fold changes of microRNA abundance in CDC exosomes compared to normal human dermal fibroblasts (NHDF) exosomes (n=4 independent experiments). Total RNA (including microRNAs) was isolated from CDC exosomes and NHDF exosomes. qRT-PCR was performed on an microRNA array. (B) Venn diagram showing the variable microRNA profile between CDC and NHDF exosomes. Font size reflects the magnitude of differential expression of each microRNA.

FIG. 3. Isolation of Exosomes from CDCs. (A) Graphical representation of exosome isolation and purification for exosomes. (B) Cell viability (calcein) and cell death (Ethidium homodimer-1) assay performed on CDCs over the 15 day serum-free conditioning period. (C) Representative images of CDCs before and after serum-free conditioning.

FIG. 4. Heat Map or microRNA PCR Array Identifies Mir-146a as a Highly Differentially Expressed microRNA. Heat map showing fold regulation differential abundance data for transcripts between CDC exosomes and NHDF exosomes overlaid onto the PCR Array plate layout.

FIG. 5. Serial body weights in control (CTL) and PAH groups (CDC and Sham) through day 35

FIG. 6. Right ventricular systolic pressure (RVSP) in healthy control (CTL) and animals with pulmonary arterial hypertension 28 days following monocrotaline subcutaneous injection (60 mg/kg) and intravenous infusion of phosphate buffered saline (PBS; sham surgery) or 2 million cardiac-derived cells (CDC) in PBS on day 14. Values are means±SEM. * significantly different from CTL. n equals 10 animals/group

FIG. 7. Right ventricular systolic pressure (RVSP) in healthy control (CTL; n=10) animals and animals with pulmonary arterial hypertension 35 days following monocrotaline subcutaneous injection (60 mg/kg) and intravenous infusion of phosphate buffered saline (PBS; sham surgery; n=9) or 2 million cardiac-derived cells (CDC; n=10) in PBS on day 14. Values are means±SEM. * significantly different from CTL; # significantly different from PBS.

FIG. 8. Fulton Index of RV hypertrophy. Fulton Index (right ventricle/[left ventricle+septum], RV/(LV+S) in healthy control (CTL) animals and animals with pulmonary arterial hypertension 28 days following monocrotaline subcutaneous injection (60 mg/kg) and intravenous infusion of phosphate buffered saline (PBS; sham surgery) or 2 million cardiac-derived cells (CDC) in PBS on day 14. Values are means±SEM. * significantly different from CTL. ;. # significantly different from PBS. NOTE: A: 28-days; B: 35 Days.

FIG. 9. Tricuspid Annular Plane systolic excursion (TAPSE), an index of RV systolic function measured at day 35. Values depicted as means±SEM.

FIG. 10. Mean vessel wall thickness for the 3 groups. Values depicted as means±SEM. * significantly different from CTL; # significantly different from PBS The mean diameters of the vessels analyzed were similar across the groups. CTL: 39.4+/−1.2 μm (SD); PAH+PBS (Sham): 40.1+/−2.8 μm; PAH+CDC: 39.5+/−3.7 μm.

FIG. 11. Immunohistochemical depiction (for smooth muscle actin) of cross-section of individual pulmonary arterioles for each of the 3 groups. Scale bar is 25 μm

FIG. 12. Bar graphs depicting improved parameters of renal function in PAH animals who received CDCs compared to sham PAH animals who only received PBS. * significantly different from CTL; # significantly different from PAH Sham. For urea nitrogen p<0.01 for * and #. For creatinine p<0.05 for * and #.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U. S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

With the onset of pulmonary arterial hypertension (PAH), the right ventricle (RV) adapts to the increased afterload presented to it by adaptive hypertrophy (i.e. RV wall muscle thickening) in order to maintain adequate blood flow. However for unknown reasons maladaptive influences ensue, resulting in impaired RV function with progressive decompensation and failure. Further, even in the modern treatment age with 12 approved PAH-specific agents, patients exhibit progressive disease and most frequently die of RV failure. It is thus intriguing to note that, despite apparent clinical improvement on PAH-specific medications, progressive RV dysfunction has been reported. Also in patients on continuous infusions of epoprostenil, often for many years, with improved clinical status, persistent severe occlusive arteriopathy and plexiform lesions in the lung have been reported. Another study examined 68 lung specimens from patients with PAH and concluded that their results indicated that multiple features of pulmonary vascular remodeling were present in patients treated with modern PAH therapies. To date there are no specific treatments that address the many pathobiologic mechanisms underlying RV dysfunction in patients with PAH. Presently, treatment is supportive at best with the use of diuretics, inotropic therapy etc. This has led leaders in the field to emphatically plea for more work on understanding the mechanisms underlying the maladapted state and unique and different treatments for a progressively failing pump.

Cardiospheres are self-associating aggregates of cells which display certain properties of cardiomyocytes, such as the ability to “beat” in vitro. They are excitable and contract in synchrony. As obtained from heart biopsies, the cells which form the cardiospheres can be disaggregated. Cardiosphere-Derived Cells (CDCs) are easily harvested and can be readily expanded from biopsy specimens. In prior studies, 69 of 70 patients had specimens that yielded cells by method described herein, making the goal of autologous cellular cardiomyoplasty attainable. Autologous cells are highly attractive as transplant material, as a perfect genetic match presenting fewer safety concerns than allogeneic cells. A practical limitation with the use of autologous cells arises from the delay from tissue harvesting to cell transplantation. As an alternative, cell banks can be created of cardiac stem cells (CSCs) from patients with defined immunological features. These should permit matching of immunological antigens of donor cells and recipients for use in allogeneic transplantation.

From single bioptome specimens, millions of CDCs can be derived after just two passages. If biopsies were performed specifically for therapeutic purposes, the amount of starting material could easily be scaled upwards by ten-fold or more, further improving the overall cell yield. The Inventors have previously used CDCs derived from human biopsies without antigenic selection. CDCs do include a sizable population of cells that exhibit stem cell markers, and the observed regenerative ability in vivo further supports the notion that CDCs include a number of resident stem cells. Described methods for ex vivo expansion of resident stem cells for subsequent autologous transplantation may give these cell populations, the resident and the expanded, the combined ability to mediate myocardial regeneration to an appreciable degree. If so, cardiac stem cell therapy may well change our fundamental approach to the treatment of disorders of cardiac dysfunction. Further examples are found in U.S. application Ser. No. 11/666,685, Ser. No. 12/622,143, and Ser. No. 12/622,106, which are herein incorporated by reference.

In the context of pulmonary arterial hypertension (PAH), an existing cell-based therapy has focused on the endothelial cell (EC) populations lining these structures, for which replacement of decreased or dysfunctional endothelial precursor cells (EPCs) via transplantation have confirmed a role for EPC in repair of pulmonary vasculature. Further engineering of early outgrowth EPCs to overexpress endothelial nitric oxide synthase (eNOS) have been developed in an attempt to maximize synergistic benefits. Other studies have begun to explore the potential use of mesenchymal stem cells (MSCs) in PAH. While prophylactic benefits may be notable, these early positive indicators are only partially or not effective, particularly in instances of established disease. In addition, concerns over use of genetically-modified cells, or safety profile of delivered cells to human subjects will persist.

In this regard, evidence that the efficacy of various stem cells is actually due, in large part, to the local release of a variety of chemical factors suggests that the release of microvescieles such as exosomes, may be a highly preferable clinical mechanism for therapy when compared to cell differentiation and engraftment, and for treatment of established disease, not merely palliative or prophylactic measures. Therapeutic effects of stem cells via regeneration can be significantly enhanced by directly delivering exosomes produced by such stem cells as an alternative to delivering the cell themselves. Towards this end, it has been reported that in a hypoxic rodent model of PAH, administration of stem cell derived exosomes prevent the development of PAH. Although interesting, such results mirror the prophylactic effects observed in other stem cell-based approaches, but do not answer the question or whether prevention and reversal adverse arteriolar allows exosomes to provide superior advantages over the use of stem cells per se.

Without being bound by any particular theory, the Inventors believe that the therapeutic effects of stem cells can be reproduced by exosomes, and are possibly indispensable to such regenerative processes. In fact, focused application of exosomes may actually provide enhanced results for the following reasons. Firstly, the retention of delivered stem cells has been shown to be short lived. Second, the quantity of local release of exosomes from a stem cell is limited and occurs only as long as the cell is retained. Thirdly, the quantity of exosomes delivered can be much higher (i.e., high dosing of its contents). Fourth, exosomes can be readily taken up by the cells in the local tissue milieu. Fifth, issues of immunogenicity are avoided. Lastly, repeated doses of exosomes is feasible, while impractical/potentially dangerous for stem cells as they impact the microvasculature.

There is a compelling rationale for using CDCs, and their cellular products (i.e., exosomes) to treat RV dysfunction in PAH. With RV dysfunction, several mechanisms have been identified. These include RV muscle capillary rarefaction due to impaired angiogenesis, with ensuing ischemia and induction of a “hibernating” state. A metabolic shift in cardiac myocytes to glycolysis, together with mitochondrial abnormalities have also been reported. There is also abnormal RV fibrosis, oxidative stress, inflammation and cardiac myocyte apoptosis. Finally in the decompensated RV, marked reductions in IGF-1 (an anabolic growth factor) is present, as well as epigenetic aberrations, such as decreased micro RNA 133a.

There are several reasons to believe that cell therapy would benefit PAH patients with RV dysfunction, as CDCs have the potential to impact many of the mechanisms underlying the pathobiology/pathophysiology of the maladapted RV muscle. These include CDCs having significant anti-inflammatory effects, attenuating both oxidative and nitrosative stress, CDCs as anti-apoptotic and anti-fibrotic, capable of attracting endogenous stem cells to sites of vascular injury, and potently angiogenic. In other contexts, CDCs have been successfully applied in models of cardiomyopathy, CDCs infused into the coronary arteries of patients with dilated cardiomyopathy, and CDC studies in patients following myocardial infarction, thereby demonstrating a track record of safety in patients. Additionally, development and use of CDC cell therapy and exosome therapy has the potential to impact directly on the microvascular pathology in PAH, by reversing the course of the disease, as opposed to palliative or preventive measures. Such a regenerative approach would be a major breakthrough therapeutically in both addressing the abnormal vasculature and offsetting the onset of right ventricular compensation which are not addressed by the current pharmacologic tools currently employed in the treatment of this devastating condition.

Realizing these benefits requires an improved understanding of whether exosomes secreted by stem cells such as CDCs, are alone capable of reproducing therapeutic benefits of their parental cells, or possibly indispensable in these processes. Confirming the role of exosomes in such processes will allow their application in new therapeutic approaches, including “cell-free” use in subjects for which cellular transplant or administration is unavailable (e.g., late stage heart disease). Pharmacological, device-based intervention or surgery may not provide significant options for such subjects. There is a great need in the art for identifying means by which to deliver the benefits of stem cell regeneration, without resorting to mechanisms involving administration or transplant of the cell themselves.

Described herein is a method for treatment including, selecting a subject in need of treatment, administering a composition including stem cells, progenitors and/or precursors to the individual, wherein the administration of the composition treats the subject. In other embodiments, the stem cells, progenitors and/or precursors are cardiosphere-derived cells (CDCs). In certain embodiments, the subject is in need to treatment for a disease and/or condition involving tissue damage or dysfunction. In other embodiments, the disease and/or condition involving tissue damage or dysfunction is pulmonary disease. In other embodiments, the pulmonary disease is pulmonary hypertension (PH) or pulmonary arterial hypertension (PAH). In other embodiments, the disease and/or condition involving tissue damage or dysfunction is heart disease.

In other embodiments, the stem cells, progenitors and/or precursors are pluripotent stem cells (pSCs), such as embryonic stem cells (ESCs) and induced pluripotent stem cells (iPSCs) derived from any one of various somatic sources in the body such as fibroblasts, blood and hematopoietic stem cells (hSCs), immune cells, bone and bone marrow, neural tissue, among others. In other embodiments, the stem cells, progenitors and/or precursors includes hSCs, mesenchymal stem cells (MSCs), or endothelial precursor cells (EPCs). In various embodiments, the cells are stem cells, progenitors and/or precursors derived from human biopsy tissue. In various embodiments, the cells are stem cells, progenitors and/or precursors are a primary culture. In various embodiments, the cells are stem cells, progenitors and/or precursors which may constitute a cell line capable of serial passaging. In various embodiments, the CDCs are mammalian. In other embodiments, the CDCs are human. In certain embodiments, the exosomes are synthetic.

In various embodiments, administration of the stem cells, progenitors and/or precursors such as cardiosphere-derived cells (CDCs), alters gene expression in the damaged or dysfunctional tissue, improves viability of the damaged tissue, and/or enhances regeneration or production of new tissue in the individual. In other embodiments, administration of the stem cells, progenitors and/or precursors such as cardiosphere-derived cells (CDCs), includes a number of cells used in a clinically relevant dose for a cell-therapy method. For example, it has been demonstrated that as few as 3 mL/3×10⁵ CDCs, is capable of providing therapeutic benefit in intracoronary administration. In another example, the number of administered CDCs includes intracoronary 25 million CDCs per coronary artery (i.e., 75 million CDCs total). In various embodiments, the numbers of CDCs includes 1×10⁵, 1×10⁶, 1×10⁷, 1×10⁸, 1×10⁹ CDCs in a single dose. In certain instances, this may be prorated to body weight (range 100,000-1M CDCs/kg body weight total CDC dose). In various embodiments, the administration can be in repeated doses. In various embodiments, the repeated or sequentially-applied doses are provided for treatment of an acute disease and/or condition. In various embodiments, the repeated or sequentially-applied doses are provided for treatment of a chronic disease and/or condition.

In various embodiments, administration of the stem cells, progenitors and/or precursors such as cardiosphere-derived cells (CDCs), to the subject occurs through any of known techniques in the art. In some embodiments, this includes percutaneous delivery, and/or injection into heart or skeletal muscle. In other embodiments, myocardial infusion is used, for example, the use of intracoronary catheters. In various embodiments, delivery can be intra-arterial or intravenous. Additional delivery sites include any one or more compartments of the heart, such as arterial, venous, and/or ventricular locations. In certain embodiments, administration can include delivery to a tissue or organ site that is different from the site or diseased and/or dysfunctional tissue. In certain embodiments, the delivery is via inhalation or oral administration.

In various embodiments, administration of the stem cells, progenitors and/or precursors such as cardiosphere-derived cells (CDCs), alters gene expression in the damaged or dysfunctional tissue, improves viability of the damaged tissue, and/or enhances regeneration or production of new tissue in the individual. In various embodiments, administration of the of the stem cells, progenitors and/or precursors such as cardiosphere-derived cells (CDCs), results in functional improvement in the tissue. In certain embodiments, the damaged tissue is pulmonary, arterial or capillary tissue. In several embodiments, the damaged or dysfunctional tissue includes cardiac tissue.

For example, in certain embodiments in which pulmonary, arterial, capillary, or cardiac tissue is damaged or dysfunctional, functional improvement may comprise increased cardiac output, contractility, ventricular function and/or reduction in arrhythmia (among other functional improvements). For example, this may include a decrease in right ventricle systolic pressure. For other tissues, improved function may be realized as well, such as enhanced cognition in response to treatment of neural damage, improved blood-oxygen transfer in response to treatment of lung damage, improved immune function in response to treatment of damaged immunological-related tissues. In other embodiments, the disease and/or condition involving tissue damage or dysfunction is pulmonary tissue, including pulmonary, arterial or capillary tissue, such as the endothelial lining of distal pulmonary arteries. In other embodiments, the pulmonary disease is pulmonary hypertension (PH) or pulmonary arterial hypertension (PAH). In some embodiments, PAH is idiopathic. In other embodiments, PAH is associated. In other embodiments, the disease and/or condition involving tissue damage or dysfunction is heart disease.

For example, in certain embodiments in which skeletal muscle tissue is damaged or dysfunctional, functional improvement may include increased contractile strength, improved ability to walk (for example, and increase in the six-minute walk test results), improved ability to stand from a seated position, improved ability to sit from a recumbent or supine position, or improved manual dexterity such as pointing and/or clicking a mouse.

In various embodiments, the damaged or dysfunctional tissue is in need of repair, regeneration, or improved function due to an acute event. Acute events include, but are not limited to, trauma such as laceration, crush or impact injury, shock, loss of blood or oxygen flow, infection, chemical or heat exposure, poison or venom exposure, drug overuse or overexposure, and the like. In certain embodiments, the damaged tissue is pulmonary, arterial or capillary tissue, such as the endothelial lining of distal pulmonary arteries. In other embodiments, the damaged tissue is cardiac tissue and the acute event includes a myocardial infarction. In some embodiments, administration of the exosomes results in an increase in cardiac wall thickness in the area subjected to the infarction.

In other embodiments, the damaged or dysfunctional tissue is due to chronic disease, such as for example congestive heart failure, including as conditions secondary to diseases such as emphysema, ischemic heart disease, hypertension, valvular heart disease, connective tissue diseases, HIV infection, liver disease, sickle cell disease, dilated cardiomyopathy, infection such as Schistosomiasis, diabetes, and the like. In various embodiments, the administration can be in repeated doses, such as two, three, four, four or more sequentially-applied doses. In various embodiments, the repeated or sequentially-applied doses are provided for treatment of an acute disease and/or condition. In various embodiments, the repeated or sequentially-applied doses are provided for treatment of a chronic disease and/or condition.

Other sources of damage also include, but are not limited to, injury, age-related degeneration, cancer, and infection. In several embodiments, the regenerative cells are from the same tissue type as is in need of repair or regeneration. In several other embodiments, the regenerative cells are from a tissue type other than the tissue in need of repair or regeneration. In other embodiments, lower pulmonary pressures, reduced right ventricular wall thickness, and/or reduction in lesion size are all indicative of treating damaged or dysfunctional tissue.

In certain embodiments, the method of treatment includes, selecting a subject in need of treatment for a pulmonary disease and/or condition, administering a composition including stem cells, progenitors and/or precursors, to the individual, wherein the administration of the composition treats the subject. In other embodiments, the stem cells, progenitors and/or precursors such as cardiosphere-derived cells (CDCs). In certain embodiments, the method of treatment includes, selecting a subject in need of treatment for a heart related disease and/or condition, administering a composition including stem cells, progenitors and/or precursors such as cardiosphere-derived cells (CDCs), to the individual, wherein the administration of the composition treats the subject. In various embodiments, administering a composition includes multiple dosages of the composition. In various embodiments, the repeated or sequentially-applied doses are provided for treatment of an acute disease and/or condition. In various embodiments, the repeated or sequentially-applied doses are provided for treatment of a chronic disease and/or condition. In other embodiments, administering a composition includes myocardial infusion. In other embodiments, administering a composition includes use of a intracoronary catheter. In other embodiments, administration of a composition includes intra-arterial infusion. In other embodiments, administration of a composition includes intravenous infusion. In other embodiments, administering a composition includes percutaneous injection, and/or injection into heart or skeletal muscle.

Further described herein is method of treatment, including selecting a subject in need of treatment for a heart related disease and/or condition; and administering a composition including cardiosphere-derived cells (CDCs), wherein the administration of the composition treats the subject. In various embodiments, the heart related disease and/or condition includes pulmonary hypertension (PH) or pulmonary arterial hypertension (PAH). In various embodiments, the PAH is idiopathic. In various embodiments, the PAH is associated. In various embodiments, administering a composition includes intra-arterial infusion. In various embodiments, administering a composition includes intravenous infusion. In various embodiments, administering a composition includes injection. In various embodiments, injection includes percutaneous injection. In various embodiments, injection includes injection into heart or skeletal muscle. In various embodiments, administering a composition includes inhalation.

Further described herein is a method of improving cardiac performance in a subject including, selecting a subject, administering a composition including stem cells, progenitors and/or precursors such as cardiosphere-derived cells (CDCs) to the individual, wherein the administration of the composition improves cardiac performance in the subject. In some embodiments, this includes a decrease in right ventricle systolic pressure. In other embodiments, there is a reduction in arteriolar narrowing, or pulmonary vascular resistance. In other embodiments, improving cardiac performance can be demonstrated, by for example, improvements in baseline ejection volume. In other embodiments, improving cardiac performance relates to increases in viable tissue, reduction in scar mass, improvements in wall thickness, regenerative remodeling of injury sites, enhanced antiogenesis, improvements in cardiomyogenic effects, reduction in apoptosis, and/or decrease in levels of pro-inflammatory cytokines. In other embodiments, lower pulmonary pressures, reduced right ventricular wall thickness, and/or reduction in lesion size suggest better cardiac performance.

In certain embodiments, the method of improving cardiac performance includes, selecting a subject in need of treatment for a heart related disease and/or condition, administering a composition including stem cells, progenitors and/or precursors such as cardiosphere-derived cells (CDCs) to the individual, wherein the administration of the composition treats the subject. In various embodiments, the heart related disease and/or condition includes heart failure. In various embodiments, administering a composition includes multiple dosages. In various embodiments, the repeated or sequentially-applied doses are provided for treatment of an acute disease and/or condition. In various embodiments, the repeated or sequentially-applied doses are provided for treatment of a chronic disease and/or condition. In other embodiments, administering a composition includes percutaneous injection. In other embodiments, administering a composition includes injection in heart or skeletal muscle. In other embodiments, administering a composition includes myocardial infusion. In other embodiments, administering a composition includes use of an intracoronary catheter. In other embodiments, administration a composition includes intra-arterial or intravenous delivery.

Described herein is a method for treatment including, selecting a subject in need of treatment, administering a composition including a plurality of exosomes to the individual, wherein the administration of the composition treats the subject. In certain embodiments, the subject is in need to treatment for a disease and/or condition involving tissue damage or dysfunction. In other embodiments, the disease and/or condition involving tissue damage or dysfunction is pulmonary disease. In other embodiments, the pulmonary disease is pulmonary hypertension (PH) or pulmonary arterial hypertension (PAH). In other embodiments, the disease and/or condition involving tissue damage or dysfunction is heart disease. In other embodiments, the plurality of exosomes includes exosomes including one or more microRNAs.

In certain embodiments, the plurality of exosomes is generated by a method including providing a population of cells, and isolating a plurality of exosomes from the population of cells. In various embodiments, the cells are stem cells, progenitors and/or precursors. In other embodiments, the stem cells, progenitors and/or precursors are cardiosphere-derived cells (CDCs). In other embodiments, the stem cells, progenitors and/or precursors are pluripotent stem cells (pSCs), such as embryonic stem cells (ESCs) and induced pluripotent stem cells (iPSCs) derived from any one of various somatic sources in the body such as fibroblasts, blood and hematopoietic stem cells (hSCs), immune cells, bone and bone marrow, neural tissue, among others. In other embodiments, the stem cells, progenitors and/or precursors includes hSCs, mesenchymal stem cells (MSCs), or endothelial precursor cells (EPCs).

In various embodiments, the plurality of exosomes is isolated from the supernatants of the population of cells. This includes, for example, exosomes secreted into media conditioned by a population of cells in culture, further including cell lines capable of serial passaging. In certain embodiments, the cells are cultured in a serum-free media. In certain embodiments, the cells in culture are grown to 10, 20, 30, 40, 50, 60, 70, 80, 90, or 90% or more confluency when exosomes are isolated. In various embodiments, the plurality of exosomes includes one or more exosomes that are about 10 nm to about 250 nm in diameter, including those about 10 nm to about 15 nm, about 15 nm to about 20 nm, about 20 nm to about 25 nm, about 25 nm to about 30 nm, about 30 nm to about 35 nm, about 35 nm to about 40 nm, about 40 nm to about 50 nm, about 50 nm to about 60 nm3 about 60 nm to about 70 nm, about 70 nm to about 80 nm, about 80 nm to about 90 nm, about 90 nm to about 95 nm, about 95 nm to about 100 nm, about 100 nm to about 105 nm, about 105 nm to about 110 nm, about 110 nm to about 115 nm, about 115 nm to about 120 nm, about 120 nm to about 125 nm, about 125 nm to about 130 nm, about 130 nm to about 135 nm, about 135 nm to about 140 nm, about 140 nm to about 145 nm, about 145 nm to about 150 nm, about 150 to about 200 nm, about 200 nm to about 250 nm, about 250 nm or more.

In various embodiments, the plurality of exosomes includes one or more exosomes expressing a biomarker. In certain embodiments, the biomarkers are tetraspanins. In other embodiments, the tetraspanins are one or more selected from the group including CD63, CD81, CD82, CD53, and CD37. In other embodiments, the exosomes express one or more lipid raft associated proteins (e.g., glycosylphosphatidylinositol-anchored proteins and flotillin), cholesterol, sphingomyelin, and/or hexosylceramides. This further includes exosomes expressing the extracellular domain of membrane-bound receptors at the surface of their membrane.

In several embodiments, isolating a plurality of exosomes from the population of cells includes centrifugation of the cells and/or media conditioned by the cells. In several embodiments, ultracentrifugation is used. In several embodiments, isolating a plurality of exosomes from the population of cells is via size-exclusion filtration. In other embodiments, isolating a plurality of exosomes from the population of cells includes use of discontinuous density gradients, immunoaffinity, ultrafiltration and/or high performance liquid chromatography (HPLC).

In certain embodiments, differential ultracentrifugation includes using centrifugal force from 1000-2000×g, 2000-3000×g, 3000-4000×g, 4000-5000×g, 5000×g-6000×g, 6000-7000×g, 7000-8000×g, 8000-9000×g, 9000-10,000×g, to 10,000×g or more to separate larger-sized particles from a plurality of exosomes derived from the cells. In certain embodiments, differential ultracentrifugation includes using centrifugal force from 10,000-20,000×g, 20,000-30,000×g, 30,000-40,000×g, 40,000-50,000×g, 50,000×g-60,000×g, 60,000-70,000×g, 70,000-80,000×g, 80,000-90,000×g, 90,000-100,000×g, to 10,000×g or more to separate larger-sized particles from a plurality of exosomes derived from the cells.

In other embodiments, isolating a plurality of exosomes from the population of cells includes use of filtration or ultrafiltration. In certain embodiments, a size exclusion membrane with different pore sizes is used. For example, a size exclusion membrane can include use of a filter with a pore size of 0.1-0.5 μM, 0.5-1.0 μM, 1-2.5 μM, 2.5-5 μM, 5 or more μM. In certain embodiments, the pore size is about 0.2 μM. In certain embodiments, filtration or ultrafiltration includes size exclusion ranging from 100-500 daltons (Da), 500-1 kDa, 1-2 kDa, 2-5 kDa, 5-10 kDa, 10-25 kDa, 25-50 kDa, 50-100 kDa, 100-250 kDa, 250-500 kDa, 500 or more kDa. In certain embodiments, the size exclusion is for about 2-5 kDa. In certain embodiments, the size exclusion is for about 3 kDa. In other embodiments, filtration or ultrafiltration includes size exclusion includes use of hollow fiber membranes capable of isolating particles ranging from 100-500 daltons (Da), 500-1 kDa, 1-2 kDa, 2-5 kDa, 5-10 kDa, 10-25 kDa, 25-50 kDa, 50-100 kDa, 100-250 kDa, 250-500 kDa, 500 or more kDa. In certain embodiments, the size exclusion is for about 2-5 kDa. In certain embodiments, the size exclusion is for about 3 kDa. In other embodiments, a molecular weight cut-off (MWCO) gel filtration capable of isolating particles ranging from 100-500 daltons (Da), 500-1 kDa, 1-2 kDa, 2-5 kDa, 5-10 kDa, 10-25 kDa, 25-50 kDa, 50-100 kDa, 100-250 kDa, 250-500 kDa, 500 or more kDa. In certain embodiments, the size exclusion is for about 2-5 kDa. In certain embodiments, the size exclusion is for about 3 kDa. In various embodiments, such systems are used in combination with variable fluid flow systems.

In other embodiments, isolating a plurality of exosomes from the population of cells includes use of tangential flow filtration (TFF) systems to purify and/or concentrate the exosome fractions. In other embodiments, isolating a plurality of exosomes from the population of cells includes use of (HPLC) to purify exosomes to homogeneously sized particles. In various embodiments, density gradients as used, such as centrifugation in a sucrose density gradient or application of a discrete sugar cushion in preparation.

In other embodiments, isolating a plurality of exosomes from the population of cells includes use of a precipitation reagent. For example, a precipitation reagent, ExoQuick®, can be added to conditioned cell media to quickly and rapidly precipitate a population of exosomes. In other embodiments, isolating a plurality of exosomes from the population of cells includes use of volume-excluding polymers (e.g., polyethylene glycols (PEGs)) are used. In another embodiment, isolating a plurality of exosomes from the population of cells includes use of flow field-flow fractionation (FIFFF), an elution-based technique.

Other examples or embodiments relating to the composition and techniques involving exosomes are presented, in PCT Pub. No. WO 2014/028,493, which is fully incorporated herein by reference.

### EXAMPLE 1

### CDCs for Treatment of PAH

As described, a critical scientific and medical question is understanding whether stem cells might be helpful in not only preventing or ameliorating disease and/or conditions such as pulmonary arterial hypertension, but actually capable of treating PAH itself via regeneration and repair of damaged microvasculature. Preliminary studies by the Inventors have shown that intravenous infusion of CDCs after the onset PAH is capable of noticeable benefits, as shown via echocardiography. Such cardiosphere derived cells (CSCs) are obtained via endomyocardial biopsies from the right ventricular aspect of the interventricular septum as obtained from healthy hearts of deceased tissue donors. Cardiosphere-derived cells are derived as described previously. See Makkar et al., (2012). “Intracoronary cardiosphere-derived cells for heart regeneration after myocardial infarction (CADUCEUS): a prospective, randomized phase 1 trial.” Lancet 379, 895-904 (2012), which is fully incorporated by reference herein.

In brief, heart biopsies are minced into small fragments and briefly digested with collagenase. Explants were then cultured on 20 mg/ml fibronectin-coated dishes. Stromal-like flat cells and phase-bright round cells grow out spontaneously from tissue fragments and reach confluence by 2-3 weeks. These cells are harvested using 0.25% trypsin and cultured in suspension on 20 mg/ml poly d-lysine to form self-aggregating cardiospheres. cardiosphere-derived cells (CDCs) are obtained by seeding cardiospheres onto fibronectin-coated dishes and passaged. All cultures are maintained at 5% CO2 at 37° C., using IMDM basic medium supplemented with 20% FBS, 1% penicillin/streptomycin, and 0.1 ml 2-mercaptoethanol. See Makkar et al., (2012). “Intracoronary cardiosphere-derived cells for heart regeneration after myocardial infarction (CADUCEUS): a prospective, randomised phase 1 trial.” Lancet 379, 895-904 (2012), which is fully incorporated by reference herein.

### EXAMPLE 2

### Preliminary Results for Therapeutic Benefits of CDCs in PAH

For animal studies, using a well-established monocrotaline injury model, animals were divided into control, monocrotaline sham infusion (i.e., no cells) and monocrotaline with cardiosphere-derived cells (+CDCs). Starting body weight was controlled across cohorts.

Echo studies and final hemodynamic studies indicated that a single dose of 2 million CDCs, infused intravenously markedly attenuate the progression of PAH in the monocrotaline rat model, as shown by the decrease systolic pressure in the right ventricle when CDCs are administered. For example, right ventricular systolic pressure (RVSP, upper panel) and Fulton Index (right ventricle/[left ventricle+septum], RV/(LV+S); lower panel) in healthy control and animals with PAH 35 days following monocrotaline subcutaneous injection (60 mg/kg) and intravenous infusion of phophase buffered saline (PBS; sham surgery) or 2 million CDCs in PBS on day 14. Values are means±SEM. * significantly different from control (CTL) # significantly different from PBS.

### EXAMPLE 3

### Media Conditioning and Exosome Purification

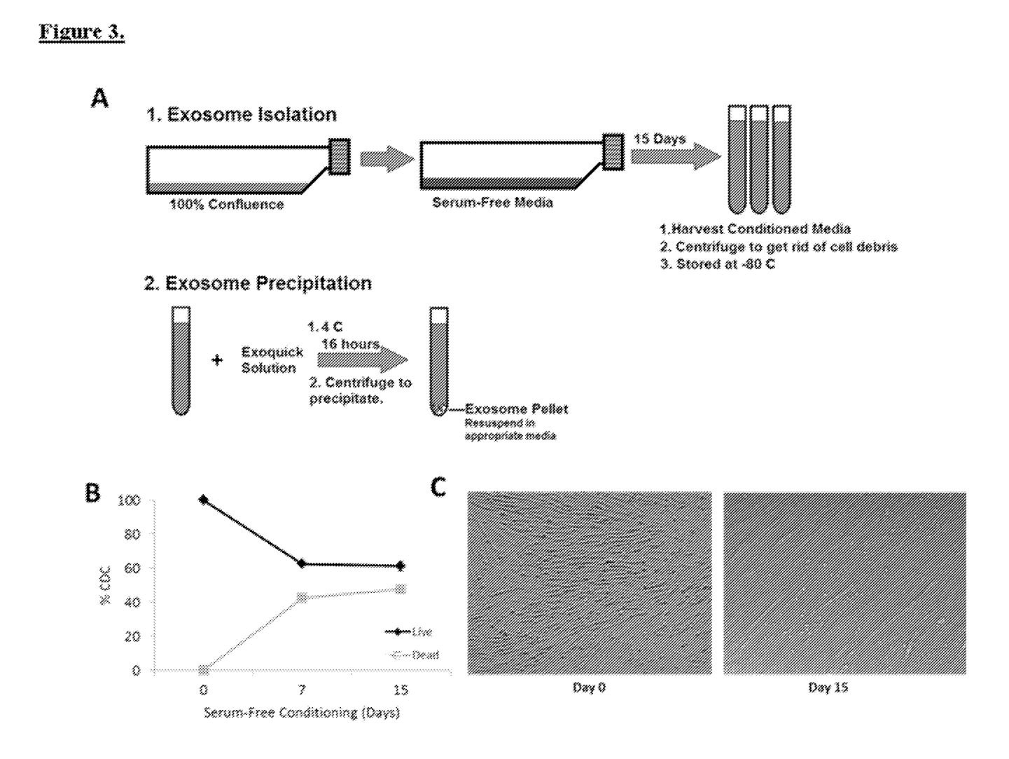

Exosomes are harvested from CDCs at passage 4. One can also isolate exosomes from normal human dermal fibroblasts (NHDF), cells that have been previously utilized as controls providing no salutary benefit, as a control. CDCs and NHDFs are conditioned in serum-free media for 15 days at 100% confluence. Aspirated media is then centrifuged at 3,000×g for 15 min to remove cellular debris. Exosomes were then isolated using Exoquick Exosome Precipitation Solution (FIG. 3).

Exosome pellets are resuspended in the appropriate media and used for assays. Expression of the conserved exosome marker CD63 is verified using ELISA. RNA content of exosome pellets can also be quantified using a Nanodrop spectrophotometer. Exosomal RNA degradation is performed by suspending exosome pellets in 2 ml of PBS. To one sample, 100 ml of Triton X-100 (Sigma Aldrich) is added to achieve 5% triton concentration. Exosomes are treated with 0.4 mg/ml RNase A treatment for 10 min at 37° C. Samples are further treated with 0.1 mg/ml Proteinase K for 20 min at 37° C. RNA is purified from samples using an microRNA isolation kit. RNA levels are measured using Nanodrop.

### EXAMPLE 4

### Mass Spectrometry Analysis on Exosome Pellets

Proteins were prepared for digestion using the filter-assisted sample preparation (FASP) method. Concentrations were measured using a Qubitfluorometer (Invitrogen). Trypsin was added at a 1:40 enzyme-to-substrate ratio and the sample incubated overnight on a heat block at 37° C. The device was centrifuged and the filtrate collected. Digested peptides were desalted using C18 stop-and-go extraction (STAGE) tips. Peptides were fractionated by strong anion exchange STAGE tip chromatography. Peptides were eluted from the C18 STAGE tip and dried. Each fraction was analyzed with liquid chromatography-tandem mass spectrometry. Samples were loaded to a 2 cm 3 100 mm I.D. trap column The analytical column was 13 cm 3 75 mm I.D. fused silica with a pulled tip emitter. The mass spectrometer was programmed to acquire, by data-dependent acquisition, tandem mass spectra from the top 15 ions in the full scan from 400 to 1,400 m/z. Mass spectrometer RAW data files were converted to MGF format using msconvert. MGF files were searched using X!Hunter against the latest spectral library available on the GPM at the time. MGF files were also searched using X!!Tandem using both the native and k-score scoring algorithms and by OMSSA. Proteins were required to have one or more unique peptides with peptide E-value scores of 0.01 or less from X!!Tandem, 0.01 or less from OMSSA, 0.001 or less and theta values of 0.5 or greater from X!Hunter searches, and protein E-value scores of 0.0001 or less from X!!Tandem and X!Hunter. Myocyte Isolation Neonatal rat cardiomyoctes (NRCMs) were isolated from 1- to 2-day-old Sprague Dawley rat pups and cultured in monolayers as described.

### EXAMPLE 5

### CDC Exosomes are Enriched in MicroRNAs Reported as Providing Provide Therapeutic Effects

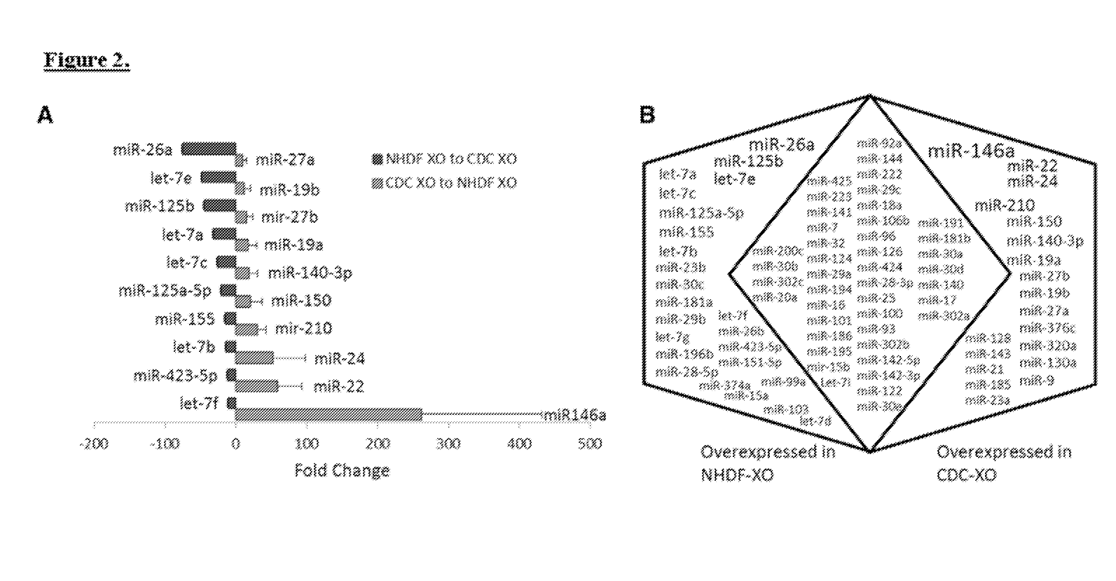

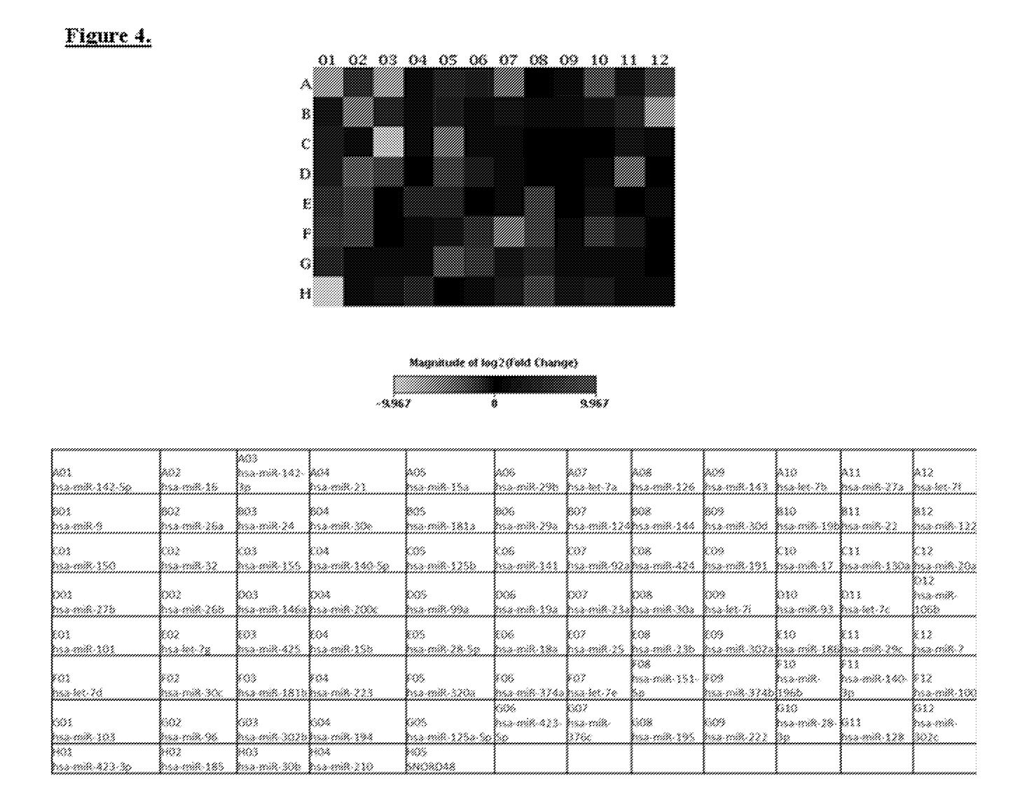

To investigate the basis of the therapeutic benefit of CDC exosomes, the Inventors compared their microRNA repertoire to that of NHDF exosomes using a PCR microarray of the 88 best-defined microRNAs. The microRNA content of the two cell types differed dramatically. Forty-three microRNAs were differentially present in the two groups; among these, miR-146a was the most highly enriched in CDC exosomes (262-fold higher than in NHDF exosomes; FIGS. 2A, 2B, and 4).

Recently, the therapeutic effects of microRNAs such as miR-146a, as derived from CDCs, have been shown as mediate some of the therapeutic benefits of CDC exosomes. For example, miR-146a leads to thicker infarct wall thickness and increased viable tissue in a mouse model of myocardial infarct. Ibrahim, et al., “Exosomes as critical agents of cardiac regeneration triggered by cell therapy.” Stem Cell Reports. 2014 May 8; 2(5):606-19, which is fully incorporated by reference herein.

### EXAMPLE 6

### CDC Exosomes for Use in PAH Therapies

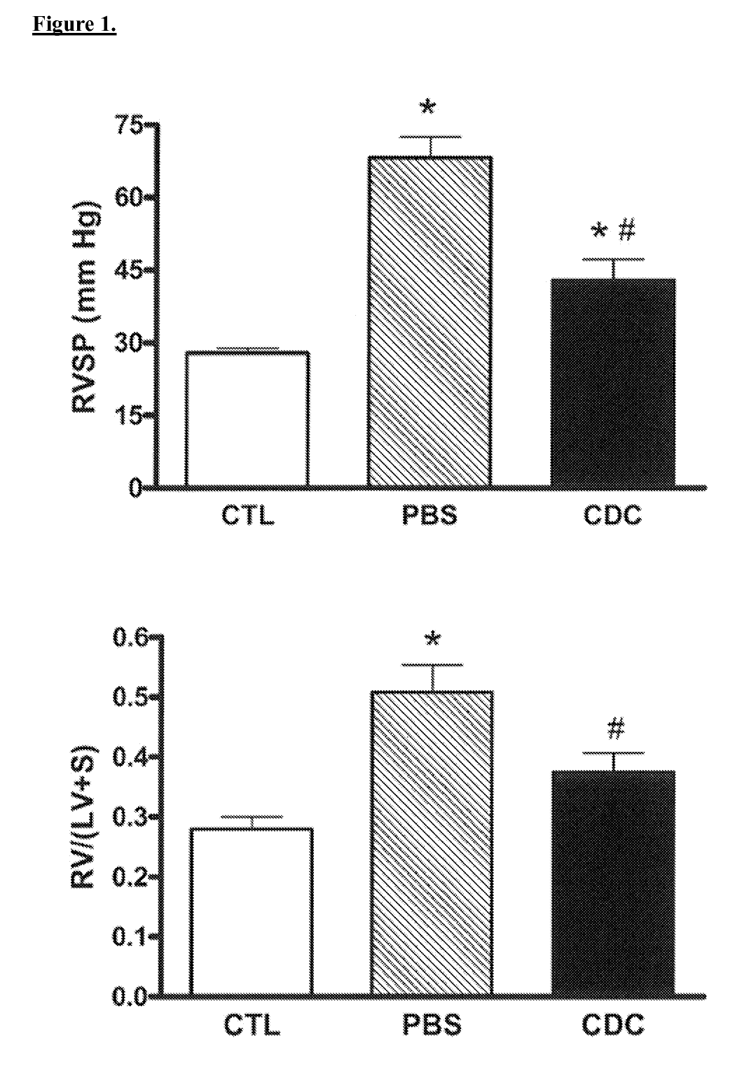

Based on the findings in Ibrahim et al, wherein CDC-derived exosomes mimic the salient benefits of CDCs, the results demonstrated in FIG. 1 confirm that CDCs work to blunt PAH. By extension, the salutary benefits of CDCs in PAH indicate that CDC-exosomes, containing their unique milieu of biological cargo, including microRNAs, will serve to replicate the therapeutic effects of CDCs in pulmonary-related conditions such as PAH.

### EXAMPLE 7

### Discussion

Cardiosphere-derived cells have been shown to induce therapeutic regeneration of the infarcted human heart. In a form of injury traditionally thought to be irreversible, CDCs led to shrinkage of scar and growth of new, functional myocardium. Similar effects have been corroborated in animal models. Here, the Inventors show that exosomes reproduce CDC-induced therapeutic regeneration, and that inhibition of exosome production undermines the benefits of CDCs. Exosomes contain microRNAs, which have the ability to alter cell behavior through paracrine mechanisms.

MicroRNAs, such as miR-146a appear to play an important part in mediating the effects of CDC exosomes, but alone may not suffice to confer comprehensive therapeutic benefit. Other microRNAs in the repertoire may exert synonymous or perhaps synergistic effects with miR-146a. For instance, miR-22 (another microRNA highly enriched in CDC exosomes) has been shown to be critical for adaptive responses to cardiac stress. Likewise, miR-24 (also identified in CDC exosomes) modulates cardiac fibrosis by targeting furin, a member of the profibrotic TGF-b signaling pathway; overexpression of miR-24 in a model of MI decreased myocardial scar formation. The possible roles of these microRNAs as mediators of CDC exosome benefits, alone or in combination with miR-146a, remain to be studied.

### EXAMPLE 7

### Identification of Cardiosphere Derived Cells as a Therapeutic Candidate for Treatment of Pulmonary Arterial Hypertension (PAH)

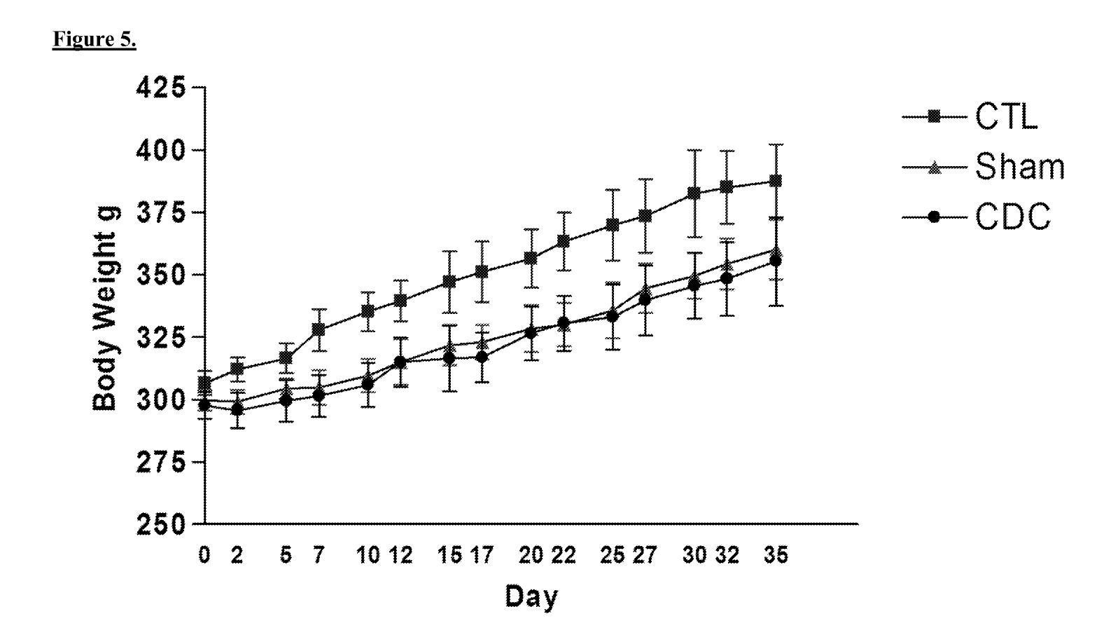

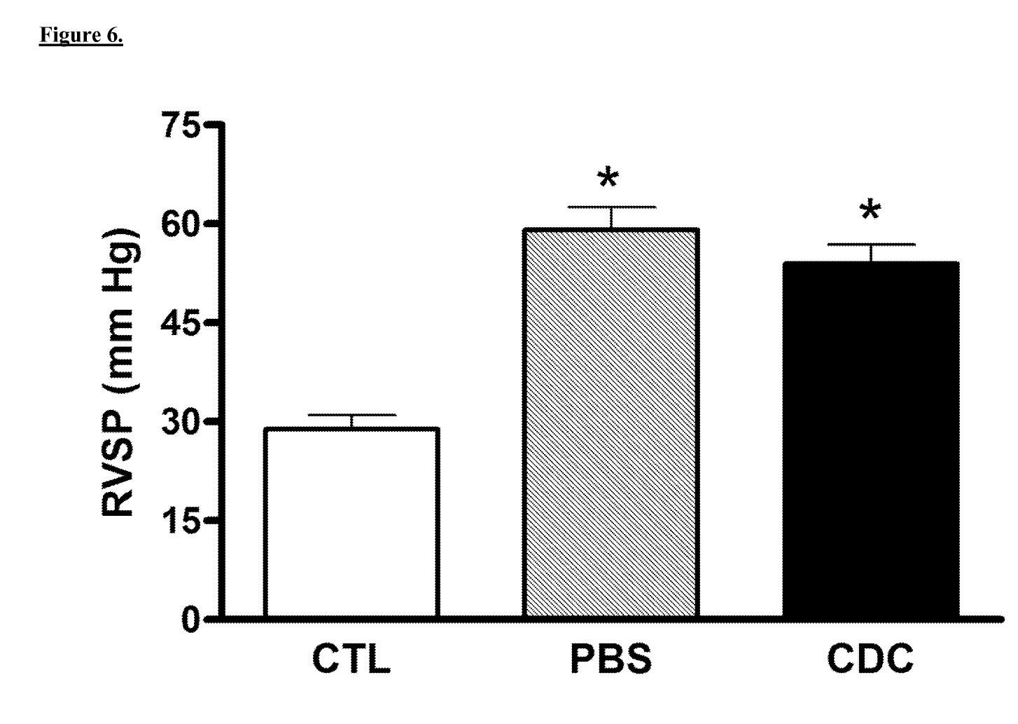

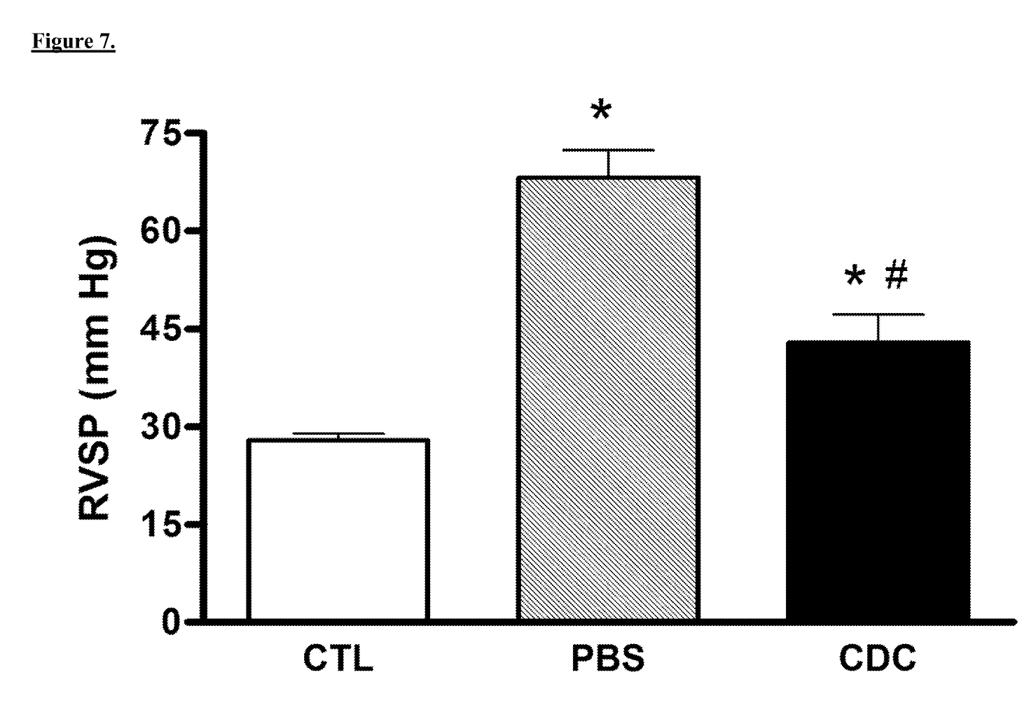

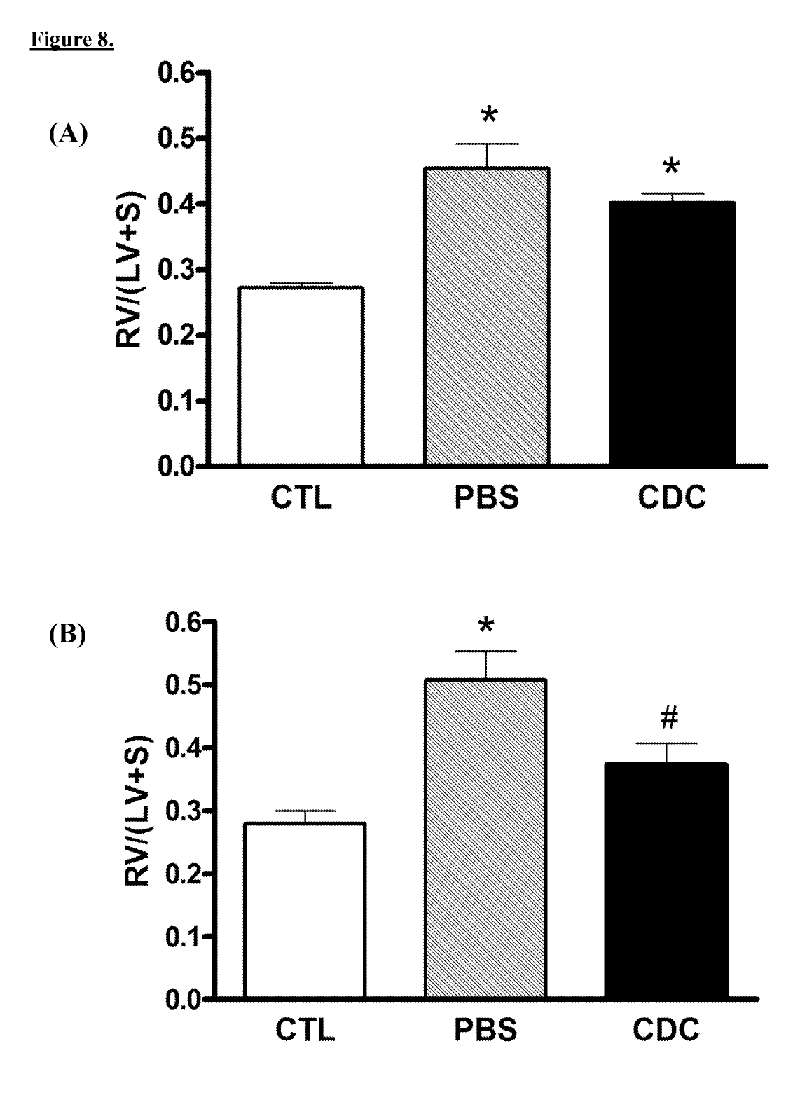

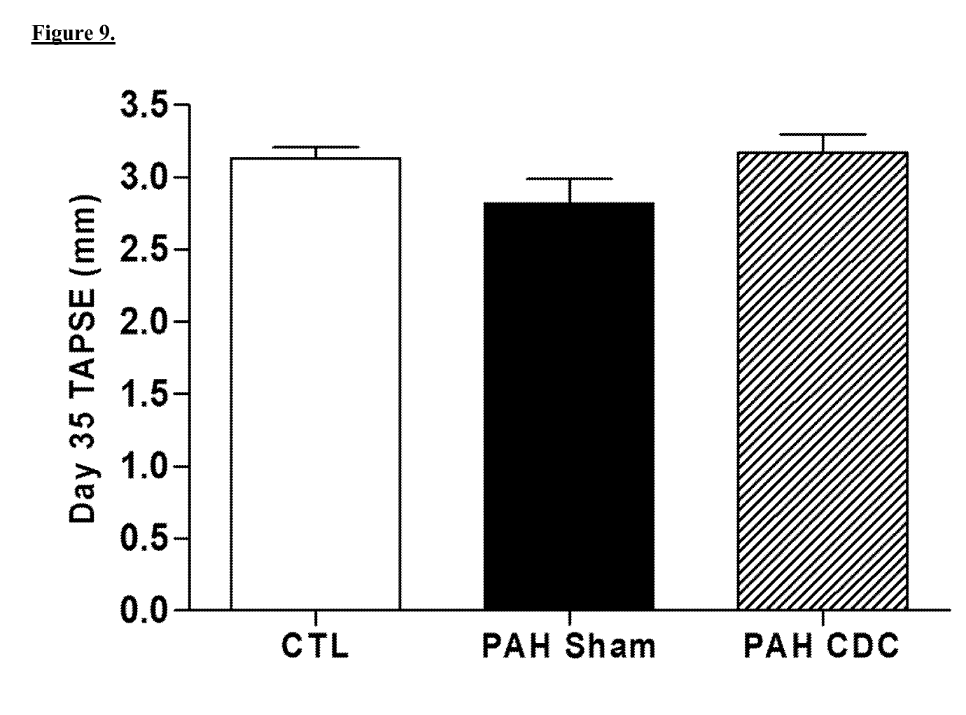

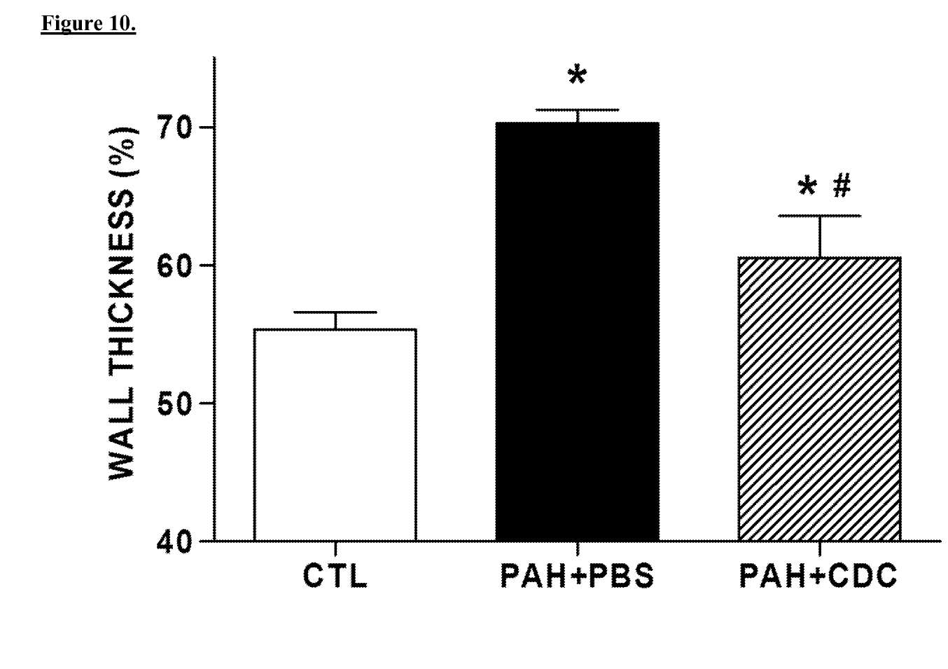

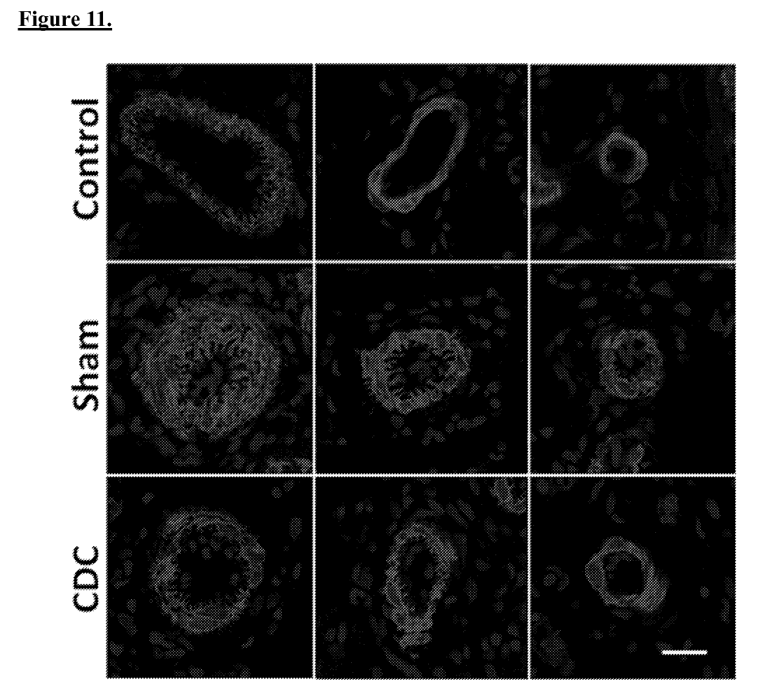

In animal studies, serial body weights in control (CTL) and PAH groups (CDC and Sham) through day 35 were measured (FIG. 5). Right ventricular systolic pressure (RVSP) was measured in healthy control (CTL) and animals with pulmonary arterial hypertension 28 days following monocrotaline subcutaneous injection (60 mg/kg) and intravenous infusion of phosphate buffered saline (sham surgery) or 2 million cardiac-derived cells (CDC) in PBS on day 14, as shown in FIG. 6. Subsequent measurement of RVSP in healthy control (CTL) animals and animals with pulmonary arterial hypertension 35 days following monocrotaline subcutaneous injection (60 mg/kg) and intravenous infusion of phosphate buffered saline (sham surgery or 2 million cardiac-derived cells (CDC; n=10) in PBS on day 14, shown in FIG. 7. As a measure of right ventricle (RV) hypertrophy, Fulton index measurement (right ventricle/[left ventricle+septum], RV/(LV+S) in healthy control (CTL) animals and animals with pulmonary arterial hypertension 28 days following monocrotaline subcutaneous injection (60 mg/kg) and intravenous infusion of phosphate buffered saline (sham surgery) or 2 million cardiac-derived cells (CDC) in PBS on day 14 are shown in 28 and 35 day measurements (FIG. 8). Resulting Tricuspid Annular Plane systolic excursion (TAPSE), an index of RV systolic function, was measured at day 35 (FIG. 9) in addition to mean vessel wall thickness for the 3 groups, CTL, PAH+PBS (Sham), PAH+CDC all possessing mean diameters of the vessels that were similar across the groups. (FIG. 10). Immunohistochemical depiction (for smooth muscle actin) of cross-section of individual pulmonary arterioles for each of the 3 groups is shown (FIG. 11).

### EXAMPLE 8

### Safety Studies

Measurement of arterial blood gas, blood and plasma and biochemistry is presented in Tables 1-3.

Arterial blood gases were drawn 24 hours post infusion of cells under general anesthesia on room air (RA). There was no significant hypoxemia at any dose of CDC.

TABLE 1
Arterial Blood Gas (ABG)

| Number | | PCO2 | PO2 | HCO3 | SaO2 |
|---|---|---|---|---|---|
| of CDCs | pH | (mmHg) | (mmHg) | (mmol/L) | (%) |
| 0.5M | 7.36 | 51.8 | 79 | 29.5 | 95 |
| 0.5M | 7.42 | 42.8 | 96 | 27.8 | 98 |
| 1M | 7.33 | 55.7 | 76 | 29.5 | 94 |
| 2M | 7.41 | 43.6 | 95 | 27.1 | 97 |
| 2M | 7.46 | 39.7 | 86 | 28.3 | 97 |
| 2M | 7.39 | 44.8 | 73 | 28.0 | 94 |
| 2M | 7.48 | 36.7 | 90 | 27.1 | 98 |

Normal ABGs in rats (n = 106) under general anesthesia are reported as: pH: 7.33 +/− 0.07; PCO2: 47.14 +/− 9.24 and PO2: 95.14 +/− 14.42

TABLE 2
Blood and Plasma Studies

| | | | | Platelet |
|---|---|---|---|---|
| | WBC | Hemoglobin | Hematocrit | Count |
| | CMM | MG/DL | % | 1000/UL |
| CTL | 5.20 | 12.2 | 39.8 | 595 |
| | 6.40 | 11 | 35.7 | 646 |
| | 3.90 | 13 | 41.8 | 641 |
| Mean | 5.17 | 12.07 | 39.10 | 627 |
| SD | 1.25 | 1.01 | 3.11 | 28 |
| PAH Sham | 7.00 | 13.7 | 42 | 655 |
| | 9.80 | 14 | 43.5 | 465 |
| | 9.30 | 14.2 | 44.5 | 534 |
| Mean | 8.70 | 13.97 | 43.33 | 551 |
| SD | 1.49 | 0.25 | 1.26 | 96 |
| PAH CDC | 8.60 | 14.4 | 45.1 | 661 |
| | 7.00 | 13.9 | 43.7 | 607 |
| | 5.40 | 13 | 42.5 | 752 |
| Mean | 7.00 | 13.77 | 43.77 | 673 |
| SD | 1.60 | 0.71 | 1.30 | 73 |

Metabolic profiling was performed at days 28 and 35. Metabolic profile of CTL, PAH Sham and PAH CDC at day 28 post MCT. No major differences were noted between CTL, PAH Sham and PAH CDC at day 28 post MCT. Chemistries reflecting renal and hepatic function at day 35 are depicted below in the table. Of note BUN and creatinine are significantly lower in the PAH CDC group compared to the PAH Sham animals. With CDCs, renal and hepatic functions are preserved and reflect control values. See Table 3.

TABLE 3
Metabolic Assays for Day 35

| | | Urea | | Bilirubin, | Total | | | |
|---|---|---|---|---|---|---|---|---|
| | Glucose | Nitrogen | Creatinine | Total | Protein | Albumin | AST | ALT |
| | MG/DL | MG/DL | MG/DL | MG/DL | G/DL | G/DL | U/L | U/L |
| CTL | 362.00 | 21.00 | 0.40 | 0.2 | 7.2 | 4.5 | 82 | 84 |
| | 367.00 | 21.00 | 0.30 | 0.2 | 5.7 | 3.7 | 67 | 71 |
| | 377.00 | 20.00 | 0.30 | 0.2 | 5.4 | 3.6 | 84 | 74 |
| | 313.00 | 23.00 | 0.30 | 0.2 | 5.3 | 3.5 | 62 | 47 |
| | 393.00 | 20.00 | 0.40 | 0.2 | 5.4 | 3.6 | 79 | 68 |
| | 402.00 | 22.00 | 0.40 | 0.2 | 7.2 | 4.5 | 78 | 58 |
| MEAN | 370.40 | 21.17 | 0.35 | 0.20 | 6.03 | 3.90 | 75.33 | 67.00 |
| SD | 34.85 | 1.17 | 0.05 | 0.00 | 0.91 | 0.47 | 8.80 | 12.93 |
| PAH | 225.00 | 59.00 | 0.60 | 0.2 | 4.9 | 2.9 | 135 | 68 |
| Sham | 288.00 | 23.00 | 0.40 | 0.2 | 5.4 | 3.6 | 77 | 45 |
| | 366.00 | 48.00 | 0.70 | 0.2 | 7 | 4.1 | 90 | 77 |
| | 323.00 | 45.00 | 0.60 | 0.2 | 6.6 | 4 | 95 | 70 |
| | 301.00 | 26.00 | 0.30 | 0.2 | 5.4 | 3.6 | 100 | 82 |
| | 430.00 | 28.00 | 0.50 | 0.2 | 6.9 | 4.3 | 76 | 75 |
| MEAN | 322.17 | 38.17 | 0.52 | 0.20 | 6.03 | 3.75 | 95.50 | 69.50 |
| SD | 70.15 | 14.55 | 0.15 | 0.00 | 0.90 | 0.50 | 21.60 | 13.00 |
| PAH | 346.00 | 23.00 | 0.40 | 0.2 | 5.6 | 3.8 | 91 | 66 |
| CDC | 394.00 | 25.00 | 0.50 | 0.2 | 6.7 | 4.2 | 105 | 100 |
| | 285.00 | 21.00 | 0.30 | 0.2 | 5.8 | 3.7 | 64 | 60 |
| | 352.00 | 23.00 | 0.40 | 0.2 | 5 | 3.4 | 73 | 66 |
| | 349.00 | 26.00 | 0.40 | 0.2 | 7 | 4.4 | 73 | 72 |
| | 324.00 | 18.00 | 0.30 | 0.2 | 5.6 | 3.6 | 87 | 76 |
| MEAN | 341.67 | 22.67 | 0.38 | 0.20 | 5.95 | 3.85 | 82.17 | 73.33 |
| SD | 35.89 | 2.88 | 0.08 | 0.00 | 0.75 | 0.38 | 14.97 | 14.18 |

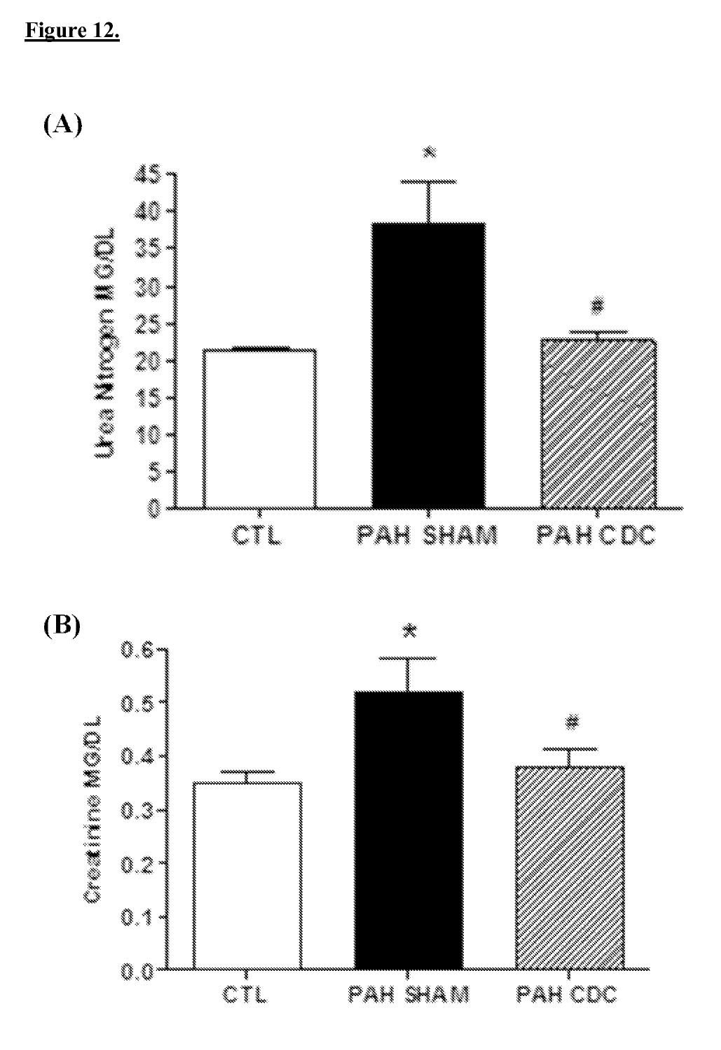

Bar graphs depicting improved parameters of renal function in PAH animals who received CDCs compared to sham PAH animals who only received PBS is shown in FIG. 12.

Based on the results described herein, CDCs are demonstrated as capable of treating pulmonary and heart-related conditions, such as pulmonary hypertension (PH) and pulmonary arterial hypertension (PAH).

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are sources of cardiosphere derived cells, the use of alternative sources such as cells derived directly from heart biopsies (explant-derived cells), or from self-assembling clusters of heart-derived cells (cardiospheres), endothelial precursor cells (EPCs) and/or mesenchymal stem cells (MSCs), exosomes produced by such cells, method of isolating, characterizing or altering exosomes produced by such cells, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of treatment, comprising:
selecting a subject in need of treatment for a heart related disease and/or condition; and administering a composition comprising cardiosphere-derived cells (CDCs), wherein the administration of the composition treats the subject.

2. The method of claim 1, wherein the heart related disease and/or condition comprises pulmonary hypertension (PH) or pulmonary arterial hypertension (PAH).

3. The method of claim 1, wherein the PAH is idiopathic.

4. The method of claim 1, wherein the PAH is associated.

5. The method of claim 1, wherein the subject has a chronic disease and/or condition.

6. The method of claim 5, wherein single dose is administered multiple times to the subject.

7. The method of claim 1, wherein administering a composition comprises intra-arterial infusion.

8. The method of claim 1, wherein administering a composition comprises intravenous infusion.

9. The method of claim 1, wherein administering a composition comprises injection.

10. The method of claim 9, wherein injection comprises percutaneous injection

11. The method of claim 9, wherein injection comprises injection into heart muscle.

12. The method claim 1, wherein treatment results in reduced ventricular wall thickness.

13. The method of claim 1, wherein treatment results in reduced pulmonary pressure or systolic pressure.

14. A method of treatment, comprising:
selecting a subject in need of treatment for a heart related disease and/or condition; and administering a composition comprising stem cells, progenitors and/or precursors to the subject, wherein the administration of the composition treats the subject.

15. The method of claim 14, wherein the heart related disease and/or condition comprises pulmonary hypertension (PH) or pulmonary arterial hypertension (PAH).

16. The method of claim 15, wherein the PAH is idiopathic.

17. The method of claim 15, wherein the PAH is associated.

18. The method of claim 14, wherein the stem cells, progenitors and/or precursors comprise cardiosphere-derived cells (CDCs).

19. The method of claim 14, wherein the subject has a chronic disease and/or condition.

20. The method of claim 19, wherein single dose is administered multiple times to the subject.

21. The method of claim 14, wherein administering a composition comprises intra-arterial infusion.

22. The method of claim 14, wherein administering a composition comprises intravenous infusion.

23. The method of claim 14, wherein administering a composition comprises injection.

24. The method of claim 23, wherein injection comprises percutaneous injection.

25. The method of claim 23, rein injection comprises injection into heart muscle.

26. The method claim 14, wherein treatment results in reduced ventricular wall thickness.

27. The method of claim 14, wherein treatment results in reduced pulmonary pressure or systolic pressure.
